(19) United States
(12) Patent Application Publication
(10) Pub. No.: US 2018/0154092 A1
(43) Pub. Date: Jun. 7, 2018

(54) DISPENSING MECHANISM

(71) Applicant: Philip Morris Products S.A., Neuchatel (CH)

(72) Inventor: Veronique PATORET, Joux (FR)

(73) Assignee: Philip Morris Products S.A., Neuchatel (CH)

(21) Appl. No.: 15/578,814

(22) PCT Filed: Jun. 8, 2016
(86) PCT No.: PCT/EP2016/063049

§ 371 Date: Dec. 1, 2017

(30) Foreign Application Priority Data
Jun. 12, 2015 (CH) 15171955.6

(51) Int. Cl. A61M 15/00; A24F 47/00; A61M 15/06

(52) U.S. Cl. CPC A61M 15/0015; A61M 15/06; A24F 47/008

(57) ABSTRACT

A dispensing mechanism for an aerosol-generating article is provided, including an expandable buffering reservoir with an inlet and an outlet; a dispensing reservoir of constant volume with an inlet and an outlet; an inlet valve connected to the inlet of the expandable buffering reservoir and being configured to be connected to a liquid storage portion filled with pressurized liquid such that liquid can only flow from the liquid storage portion to the expandable buffering reservoir; an outlet valve connecting the outlet of the expandable buffering reservoir to the inlet of the dispensing reservoir such that the liquid can only flow from the expandable buffering reservoir to the dispensing reservoir; and a volume modifier configured to modify a volume of the expandable buffering reservoir.

The present invention relates to dispensing mechanisms for aerosol-generating articles that preferably comprise a liquid storage portion filled with liquid and a vaporiser where the liquid is vaporised.

Such articles usually use a wick or foam impregnated of liquid to draw the liquid to the vaporiser. With such a design, several key factors of the smoking experiences cannot be correctly controlled, in particular the quantity of liquid that will be vaporised during one puff, i.e. during one draw of air.

This quantity changes according to the concentration of liquid in the foam or wick as the foam or wick may be more or less impregnated or dried. The quantity of liquid that will be vaporised during one puff is also influenced by the duration of the drawing of air as the vaporiser is activated and vaporises liquid as long as the user draws air. The quantity also depends on the age of the wick or foam as the capillary properties may change over time. Furthermore, the kind of movements and gestures of a user before drawing air have an impact on the quantity of liquid in the foam or wick. This impact is at least partially related to gravity and the filling level of the liquid storage portion, and thus causing the wick or foam of being more or less impregnated.

The quantity of nicotine delivered per puff is directly related to the quantity of liquid being vaporised. As the quantity of liquid to be vaporised cannot be determined or efficiently limited, the user experience could be different from one puff to another and from similar products to another. Another point is that, with such a system, the number of puffs that can be attributed to a full liquid storage portion cannot be well determined in advance, although it could be a strong marketing feature.

WO 2014/150131 A1 discloses an electronic smoking article comprising a liquid supply reservoir and an electrically operated valve that leads the liquid to a capillary tube with heaters around for vaporizing the liquid. A puff sensor detects the drawing of air in order to open the valve and to activate the heater. Upon conclusion of a puff, the valve may be closed to reduce the amount of liquid that is drawn back from the capillary tube because of the release of pressure. The amount of liquid to be vaporised during one draw of air may change from puff to puff and depends on pressure and duration.

US 2011/0120455 A1 discloses an aerosol dispensing device with a puff-controlled valve. The valve block is biased by a compression spring against a valve seat that keeps the valve closed. Upon a drawing of air exceeding the compression force of the spring, the valve opens. The amount of liquid to be vaporised during one draw of air may change from puff to puff and depends on pressure and duration.

It would be desirable to provide an improved dispensing mechanism for aerosol-generating articles that limits the amount of liquid to be vaporised during one puff, thereby guaranteeing a minimum number of puffs attributed to a full liquid storage portion, and that is based on a technically less complex mechanism.

According to a first aspect of the invention, a dispensing mechanism for an aerosol-generating article is provided, comprising an expandable buffering reservoir with an inlet and an outlet, a dispensing reservoir of constant volume with an inlet and an outlet, an inlet valve connected to the inlet of the expandable buffering reservoir, wherein the inlet valve is configured to be connected to a liquid storage portion filled with pressurized liquid such that the liquid can only flow from the liquid storage portion to the expandable buffering reservoir, an outlet valve connecting the outlet of the expandable buffering reservoir to the inlet of the dispensing reservoir such that liquid can only flow from the expandable buffering reservoir to the dispensing reservoir, and a volume modifier configured to modify the volume of the expandable buffering reservoir, wherein expanding a current volume of the expandable buffering reservoir to an expanded volume causes a drawing of liquid from the liquid storage portion to the expandable buffering reservoir until the expanded volume of the expandable buffering reservoir is filled up with the liquid, and wherein compressing the expanded volume of the expandable buffering reservoir causes an expelling of liquid from the expandable buffering reservoir to the dispensing reservoir until the dispensing reservoir is filled up with the liquid. Furthermore, the dispensing reservoir (2) is configured to release its content of liquid during one puff of a user.

According to a second aspect of the invention, an aerosol-generating article is provided, comprising a liquid storage portion configured to store pressurized liquid, a vaporiser configured to vaporise liquid, the dispensing mechanism according to the first aspect of the invention that connects the liquid storage portion to the vaporizer, and a housing having at least one air inlet and at least one air outlet, wherein the vaporiser is configured to receive liquid from the outlet of the dispensing reservoir.

According to a third aspect of the invention, an aerosol-generating system is provided, comprising the aerosol-generating article according to the second aspect of the invention, and a mouthpiece arranged to deliver the vaporised liquid from the vaporiser to a user that draws into the mouthpiece.

The expandable buffering reservoir may have a minimum volume that is expandable up to a maximum volume.

The expandable buffering reservoir and the dispensing reservoir may be configured such that the difference between the maximum volume and the minimum volume of the expandable buffering reservoir is greater or equal the constant volume of the dispensing reservoir. By doing so, the volume of the dispensing reservoir is entirely filled with liquid upon one puff.

The dispensing mechanism may further comprise a biasing member that is configured to apply a compressive force onto the volume modifier such that the expanded volume of the expandable buffering reservoir is compressed. The compression force holds the volume modifier at an initial position that corresponds to a minimum volume of the expandable buffering reservoir.

The dispensing mechanism may further comprise a dispenser connected to the outlet of the dispensing reservoir. Preferably, the dispenser is configured to open when mechanically pressed and otherwise to stay closed. More preferably, the dispenser is embodied as a deformable opening at one end of the dispensing reservoir.

The volume modifier may comprise a piston that is configured to define, together with a housing in which the dispensing mechanism is arranged, a moving chamber.

The expandable buffering reservoir may be coupled to one side of the piston and arranged in the moving chamber such that moving the piston longitudinally relative to the housing causes modifying the volume of the expandable buffering reservoir.

The dispensing reservoir may be coupled to the other side of the piston opposite to the moving chamber, thereby causing the dispensing reservoir to be moved together with the piston longitudinally in the housing.

The piston may comprise the outlet valve connecting the outlet of the expandable buffering reservoir to the inlet of the dispensing reservoir.

The biasing member may be a compression spring attached to the piston. The compression force of the compression spring pushes the moving chamber away from a vaporiser and holds the moving chamber in an initial position. Preferably, the Resistance To Draw (RTD) is controlled by changing the characteristics of the biasing member, for example by selecting a compression spring having a compression force that corresponds to the required RTD. The RTD is an important parameter of the smoking experience. The harder the spring, the stronger the user will have to draw air to put e-liquid on the vaporiser, and therefore the bigger the RTD.

The expandable buffering reservoir is deformable. Preferably, the expandable buffering reservoir is accordion-shaped, and wherein modifying the volume of the expandable buffering reservoir comprises adjusting the length of the expandable buffering reservoir. The expandable buffering reservoir is connected to an inlet valve and an outlet valve, both valves acting as one-way valves. The inlet value allows liquid only to enter into the expandable buffering reservoir and the outlet valve only to flow out of the expandable buffering reservoir. The expandable buffering reservoir may be made from a polymer, for example a plastic polymer such as PVC or PP, which is molded accordion-shaped.

The dispensing mechanism may further comprise a chained arrangement of one or more expandable drawing reservoirs, wherein the one or more expandable drawing reservoirs are arranged between the expandable buffering reservoir and the dispensing reservoir.

Each of the one or more expandable drawing reservoirs may be connected to a neighbouring reservoir by a one-way valve in such a way that liquid only flows from the expandable buffering reservoir to the dispensing reservoir.

At least a part of the one or more expandable drawing reservoirs may be attached to a side of the piston that is opposite the expandable buffering reservoir.

Preferably, the volume modifier is configured to expand the current volume of the expandable buffering reservoir to the expanded volume upon a user drawing air into the housing in which the dispensing mechanism is mounted.

Preferably, the housing comprises at least one housing stop that is configured to press or squeeze a dispenser connected to the outlet of the dispensing reservoir when the dispensing reservoir moves longitudinally relative to the housing towards the at least one housing stop upon the volume modifier expanding the volume of the expandable buffering reservoir.

The housing may comprise at least one opening between the at least one housing stop that allows the dispensing reservoir to move so that its deformable dispenser opening is next to the at least one housing stop.

Preferably, the dispensing mechanism is arranged in an aerosol-generating article with a liquid storage portion adjoining a housing of the aerosol-generating article, at the opposite side of a vaporiser. The vaporiser is arranged to vaporise liquid released by the dispenser.

The dispensing mechanism may also be arranged in an aerosol-generating device like an e-cigarette with an electronic part connected to a battery and to an embodiment of the aerosol-generating article.

Preferably, the housing has at least one air inlet and at least one air outlet such that when a user draws air into the housing, the moving chamber acts as a sliding piston and is pushed or drawn by air towards the vaporiser and moves back upon the compression force of the biasing member.

The dispensing reservoir may be configured deformable. The dispensing reservoir may be made of silicon. Alternatively or additionally, the dispensing reservoir may have a deformable opening. The deformable opening is arranged such that the content of the dispensing reservoir, preferably a liquid aerosol-forming substrate, may be released by deforming the deformable opening. The deformable opening may be mechanically deformed or squeezed as described in more detail below. The deformable opening of the dispensing reservoir may be made of a flexible material, preferably silicon, forming a baby bottle teat valve. A baby bottle teat valve is provided as a one-way valve, configured to seal the dispensing reservoir when the content of the dispensing reservoir is not pressurized and release the content of the dispensing reservoir when the content is pressurized. The dispensing reservoir may have a volume of 0.1 to 4 microliters or 0.2 to 2 microliters or around 0.4 to 0.5 microliters. Preferably, the content of liquid aerosol-forming substrate in the dispensing reservoir is completely dispensed during one puff of a user. Thus, preferably the amount of around 0.4 to 0.5 microliters of liquid aerosol-forming substrate is dispensed during one puff of a user.

The deformable opening may alternatively be configured as a duckbill valve. A duckbill valve is configured in the shape of a duckbill and is made of a flexible material such as a flexible polymer. Thus, the deformable opening may be configured having lips, preferably elastomeric lips, in the shape of a duckbill. The lips may prevent backflow and allows forward flow. In this case, the deformable opening is configured as a one-way valve. An advantage of a duckbill valve is that the duckbill valve is self-contained, i.e. seals the content of the dispensing reservoir when the content is not pressurized. Thus, leakage of the content of the dispensing reservoir may be prevented. Also, the dispensing reservoir as a whole may be in the shape of a duckbill valve or a baby bottle teat valve. Alternatively, the deformable opening of the dispensing reservoir may be configured as a two-way valve.

### First Preferred Embodiment

In a first preferred embodiment, the dispensing mechanism has two reservoirs: one expandable buffering reservoir and one dispensing reservoir.

When the user draws air into the housing with a drawing force that exceeds the compression force of the biasing member, the volume modifier is drawn towards housing stops at one end of the housing. The movement of the volume modifier elongates the expandable buffering reservoir and moves the dispensing reservoir to the housing stops where the dispensing reservoir is mechanically squeezed so that the dispensing reservoir releases its content on a vaporiser that functions as a heat source for vaporising the released liquid. The increase of volume due to the elongation of the expandable buffering reservoir causes a drawing of liquid from the liquid storage portion into the expandable buffering reservoir and refills it.

When there is no air drawn into the housing or when the drawing force is below the compression force of the biasing member, the biasing member pushes back the dispensing reservoir as well as the expandable buffering reservoir away from the vaporiser. The dispensing reservoir stops releasing liquid to the vaporiser. As only the content of the dispensing reservoir is released during one puff, the quantity of liquid, and accordingly of nicotine, that is delivered per draw is determined in advance. Therefore, the number of puffs per cartridge that acts as a liquid storage portion can be easily determined.

### Second Preferred Embodiment

In a second preferred embodiment, the dispensing mechanism has at least three reservoirs: one expandable buffering reservoir, at least one expandable drawing reservoir and one dispensing reservoir.

When the user draws air into the housing with a drawing force that exceeds the compression force of the biasing member, the volume modifier is drawn towards housing stops. The movement of the volume modifier elongates the expandable buffering reservoir and contracts the at least one expandable drawing reservoir. The movement of the volume modifier causes moving the dispensing reservoir to the housing stops where the dispensing reservoir is mechanically squeezed so that the dispensing reservoir releases its content on a vaporiser that functions as a heat source for vaporising the released liquid. The increase of volume due to the elongation of the expandable buffering reservoir causes a drawing of liquid from the liquid storage portion into the expandable buffering reservoir and refills it. The decrease of volume due to the contraction of the expandable drawing reservoir pushes liquid from the expandable drawing reservoir into the dispensing reservoir. As the dispensing reservoir is still open, the liquid is released to the vaporiser.

When there is no air drawn into the housing or when the drawing force is below the compression force of the biasing member, the biasing member pushes back the dispensing reservoir as well as the expandable drawing reservoir and the expandable buffering reservoir away from the housing stops. The dispensing reservoir stops releasing liquid to the vaporiser. The expandable drawing reservoir and the expandable buffering reservoir return to their initial position with a minimum volume of the expandable buffering reservoir and a maximum volume of the expandable drawing reservoir.

As only the volume of the dispensing reservoir plus the volume of the expandable drawing reservoir may be released during one puff, the quantity of e-liquid, and accordingly of nicotine, that is delivered per draw is limited to a fixed maximum. Therefore, a minimum number of puffs per cartridge that acts as a liquid storage portion can be easily determined in advance.

Preferably, a secured cartridge is used as liquid storage portion that is connected to the inlet valve of the dispensing mechanism. The secured cartridge comprises a chamber, a plunger and joint, a bidirectional valve, a Luer connection, a spring, and a lipido-resistant filter. The chamber of the secured cartridge contains a liquid. The chamber may contain at least one of e-liquid and flavour, pressurized by the spring. The chamber containing the liquid and the chamber of the spring are separated by the plunger and the joint. The lipido-resistant filter, allowing air but no liquid to pass, is at one end of the secured cartridge. The lipido-resistant filter assures that the pressure is equal between the spring chamber and exterior of the secured cartridge while avoiding liquid leakage. The connection with the dispensing mechanism of an aerosol-generating article is done with a Luer connection which is a standardized technology allowing an user-friendly and leak-free connection, and includes a bidirectional valve. With the bidirectional valve, the liquid inside the secured cartridge cannot flow out when the secured cartridge is not connected to the dispensing mechanism of the aerosol-generating article. When the secured cartridge is connected, the liquid can flow through the valve in and out of the secured cartridge. This structure allows to easily fill the secured cartridge with e-liquid, makes the secured cartridge easy to plug into the aerosol-generating article due to the Luer connection, and is leak proof which is a particularly important point for nicotine based e-liquid content.

Preferably, the dispensing mechanism is used in conjunction with an e-liquid cartridge as liquid storage portion, wherein the e-liquid cartridge may be connected to and disconnected from the dispensing mechanism.

Alternatively, the dispensing mechanism may also be used in conjunction with an e-liquid reservoir as liquid storage portion, wherein the user may not separate the e-liquid reservoir from the dispensing mechanism.

A plurality of dispensing systems may be arranged within one aerosol-generating article. Each dispensing system comprises a dispensing mechanism according to an embodiment of the present invention and a separate liquid storage portion.

Two dispensing systems may be arranged within one aerosol-generating article. Such an embodiment allows a user to mix two different kinds of liquid in the same e-cigarette. The user may put e-liquid into the first liquid storage portion and a specific flavour into the second liquid storage portion.

The invention will be further described, by way of example only, with reference to the accompanying drawings in which:

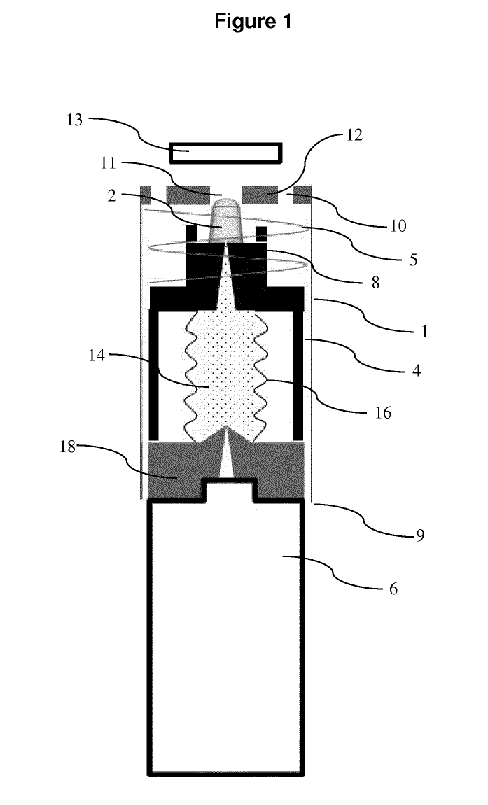

FIG. 1 shows a cross-sectional view of an aerosol-generating article with a dispensing mechanism according to a first preferred embodiment of the present invention;

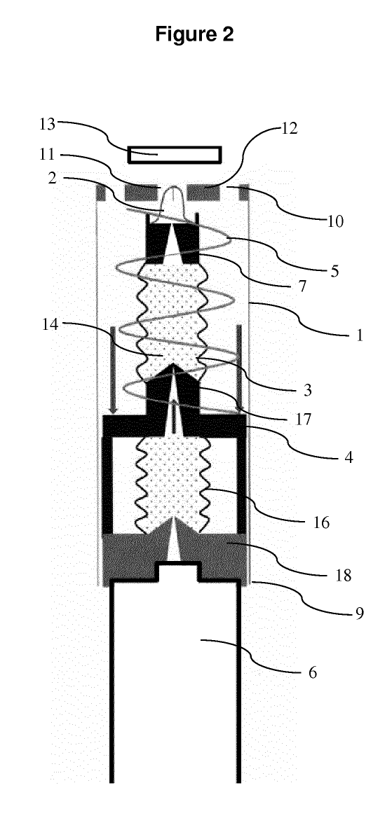

FIG. 2 shows a cross-sectional view of an aerosol-generating article with a dispensing mechanism according to a second preferred embodiment of the present invention;

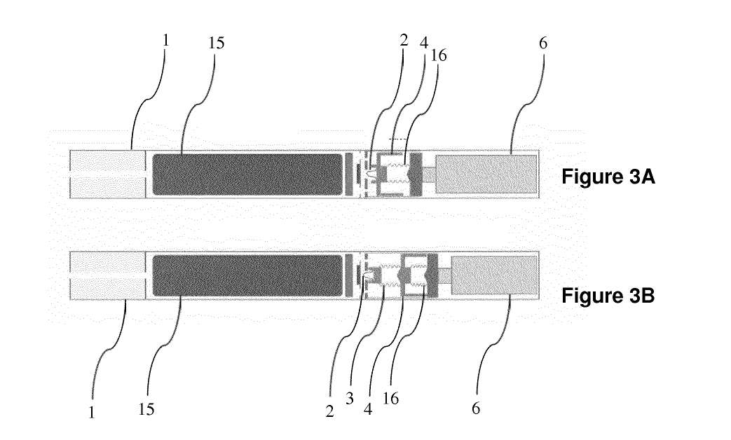

FIG. 3A shows a cross-sectional view of an aerosol-generating device with power supply, electronics, and a dispensing mechanism according to the first preferred embodiment of the present invention; and

FIG. 3B shows a cross-sectional view of an aerosol-generating device with power supply, electronics, and a dispensing mechanism according to the second preferred embodiment of the present invention.

FIG. 1 shows an aerosol-generating device embodied as an e-cigarette according to a first preferred embodiment of the present invention. The e-cigarette comprises an expandable buffering reservoir 16 of accordion shape attached to a volume modifier 4. The volume modifier 4 is embodied as a moving chamber in which the expandable buffering reservoir 16 is located. The volume modifier 4 is movable along the longitudinal axis of the e-cigarette. A biasing member 5 embodied as a compression spring is arranged between housing stops 12 and the volume modifier 4 to hold the volume modifier 4 in an initial position away from the housing stops 12. The expandable buffering reservoir 16 is connected with one-way valves 18, 8, with at one end a liquid storage portion 6 embodied as an e-liquid cartridge and at the other end a dispensing reservoir 2 with an opening, being able to move according to the moving of the moving chamber.

When a user draws air into the e-cigarette with a drawing force that exceeds the compression force of the biasing member 5, the air flow from the air inlet 9 to the air outlet 10 causes a moving of the volume modifier 4 towards the housing stops 12. Due to this movement, also the dispensing reservoir 2 is moved towards the housing stops 12, thereby compressing the biasing member 5. The opening of the dispensing reservoir 2 is pressed against the housing stops 12. Due to the mechanically squeezing of the opening of the dispensing reservoir 2 against the housing stops 12, the dispensing reservoir 2 releases its content on a vaporiser 13, directly or indirectly through a host material. The vaporiser 13 functions as a heat source for vaporising the released liquid. Simultaneously, due to the movement of the volume modifier 4 towards the housing stops 12, the length of the accordion-shaped expandable buffering reservoir 3 extends, and therefore increases its volume, which draws e-liquid from the liquid storage portion 6 via the one-way valve 8.

Once the user stops drawing air, the biasing member 5 pushes back the volume modifier 4 to its initial position. By doing so, the length, and therefore also the volume, of the accordion-shaped expandable buffering reservoir 3 decreases, forcing the expandable buffering reservoir 3 to expel its extra e-liquid drawn previously from the liquid storage portion 6 into the dispensing reservoir 2 via the one-way valve 7, filling anew the dispensing reservoir 2. Due to this movement, also the dispensing reservoir 2 moves away from the housing stops 12 so that the opening of the dispensing reservoir 2 is closed.

In this embodiment, the quantity of e-liquid that is released per puff on the vaporiser 13 is approximately equal to the quantity of e-liquid released from the dispensing reservoir 2. The quantity of e-liquid, and therefore of nicotine, may be accurately determined in advance, as well as the number of puffs that can be made per e-liquid cartridge. The number of puffs per e-liquid cartridge is approximately equal to the volume of the liquid storage portion 6 divided by the volume of the dispensing reservoir 2.

FIG. 2 shows an aerosol-generating device embodied as an e-cigarette according to a second preferred embodiment of the present invention. The e-cigarette comprises an expandable buffering reservoir 16 of accordion shape attached to a volume modifier 4. The volume modifier 4 is embodied as a moving chamber in which the expandable buffering reservoir 16 is located. The volume modifier 4 is movable along the longitudinal axis of the e-cigarette. A biasing member 5 embodied as a compression spring is arranged between housing stops 12 and the volume modifier 4 to hold the volume modifier 4 in an initial position away from the housing stops 12. The expandable buffering reservoir 16 connected with one-way valves 18, 17, with at one end a liquid storage portion 6 embodied as an e-liquid cartridge and at the other end an expandable drawing reservoir 3 of accordion shape. The expandable drawing reservoir 3 is connected with one-way valves 17, 7, with at one end the expandable buffering reservoir 16 and at the other end a dispensing reservoir 2 with an opening, being able to move according to the moving of the moving chamber.

When a user draws air into the e-cigarette with a drawing force that exceeds the compression force of the biasing member 5, the air flow from the air inlet 9 to the air outlet 10 causes of a moving of the volume modifier 4 towards the vaporiser 13, thereby compressing the biasing member 5. The movement of the volume modifier 4 also moves the expandable drawing reservoir 3 and the dispensing reservoir 2 towards the housing stops 12. The dispensing reservoir 2 is then blocked by and its opening pressed against the housing stops 12. The opening of the dispensing reservoir 2 is pressed against the housing stops 12. Due to the mechanically squeezing of the opening of the dispensing reservoir 2 against the housing stops 12, the dispensing reservoir 2 releases its content on a vaporiser 13, directly or indirectly through a host material. The vaporiser 13 functions as a heat source for vaporising the released liquid. The expandable drawing reservoir 3, pressed by the moving chamber 4 and blocked by the dispensing reservoir 2, is compressed, decreasing its length, and therefore its volume, which presses its e-liquid content out of it, towards the dispensing reservoir 2 and the housing stops 12, via the one-way valve 7. Simultaneously, the length of the expandable buffering reservoir 16 extends, and therefore does its volume, which draws e-liquid from the liquid storage portion 6 via the one-way valve 18, refilling the expandable buffering reservoir 16. In this embodiment, the expandable drawing reservoir 3 will be compressed as long as the user draws air, and its content will flow into the dispensing reservoir 2 and then, due to the fact that the opening of the dispensing reservoir 2 is still open, to the vaporiser 13.

Once the user stops drawing air, the spring 5 pushes back the volume modifier 4 in its initial position. By doing so, the length, and therefore the volume, of the expandable drawing reservoir 3 increases, drawing e-liquid from the expandable buffering reservoir 16 which in turn is compressed by the action of the biasing member 5, making the expandable buffering reservoir 16 to expel e-liquid to the expandable drawing reservoir 3.

In this embodiment, the quantity of e-liquid that is released on the vaporiser 13 depends on the duration of the air drawn by the user, but has a maximum quantity which is the volume of the dispensing reservoir 2 added to the volume of the expandable drawing reservoir 3. This embodiment allows the user to easily adjust the amount of e-liquid to be vaporised per puff. The more the user draws on the e-cigarette, the bigger the puff, while at the same time, setting a fully determined maximum quantity of e-liquid, and thus a maximum amount of nicotine is vaporised per puff.

FIG. 3A shows an aerosol-generating device embodied as an e-cigarette with power supply, electronics, and a dispensing mechanism according to FIG. 1.

FIG. 3B shows an aerosol-generating device embodied as an e-cigarette with power supply, electronics, and a dispensing mechanism according to FIG. 2.

From the foregoing, it will be appreciated that specific examples of dispensing mechanisms and aerosol-generating articles, devices, and systems have been described for the purposes of illustration, but various modifications may be made without deviating from the spirit of the present disclosure. The features of embodiments described herein may be used in combination with features of other embodiments. Embodiments described herein may be implemented without all of the features ascribed to them herein. Such variations on described embodiments that would be apparent to the skilled person, including variations that are based on a mixing of features from different embodiments, are within the scope of this invention.

### LIST OF REFERENCE SIGNS

1 housing
2 dispensing reservoir
3 expandable drawing reservoir
4 volume modifier
5 biasing member
6 liquid storage portion
7 one-way valve between drawing reservoir 3 and dispensing reservoir 2
8 one-way valve between buffering reservoir 16 and dispensing reservoir 2
9 air inlet
10 air outlet
11 opening
12 housing stops
13 vaporiser
14 liquid
15 power supply and electric circuitry
16 expandable buffering reservoir
17 one-way valve between buffering reservoir 16 and drawing reservoir 3
18 one-way valve between cartridge 6 and buffering reservoir 16

1.-19. (canceled)

20. A dispensing mechanism for an aerosol-generating article, comprising:
an expandable buffering reservoir with an inlet and an outlet;
a dispensing reservoir of constant volume with an inlet and an outlet;
an inlet valve connected to the inlet of the expandable buffering reservoir and being configured to be connected to a liquid storage portion filled with pressurized liquid such that liquid can only flow from the liquid storage portion to the expandable buffering reservoir;
an outlet valve connecting the outlet of the expandable buffering reservoir to the inlet of the dispensing reservoir such that the liquid can only flow from the expandable buffering reservoir to the dispensing reservoir; and
a volume modifier configured to modify a volume of the expandable buffering reservoir,
wherein expanding a current volume of the expandable buffering reservoir to an expanded volume causes a drawing of liquid from the liquid storage portion to the expandable buffering reservoir until the expanded volume of the expandable buffering reservoir is filled up with the liquid,
wherein compressing the expanded volume of the expandable buffering reservoir causes an expelling of the liquid from the expandable buffering reservoir to the dispensing reservoir until the dispensing reservoir is filled up with the liquid, and
wherein the dispensing reservoir is configured to release a content of the liquid during one puff of a user.

21. The dispensing mechanism of claim 20,
wherein the expandable buffering reservoir has a minimum volume that is expandable up to a maximum volume, and
wherein the expandable buffering reservoir and the dispensing reservoir are configured such that a difference between the maximum volume and the minimum volume of the expandable buffering reservoir is greater than or equal to the constant volume of the dispensing reservoir.

22. The dispensing mechanism of claim 20, further comprising a biasing member configured to apply a compressive force onto the volume modifier such that the expanded volume of the expandable buffering reservoir is compressed.

23. The dispensing mechanism of claim 20, further comprising a dispenser connected to the outlet of the dispensing reservoir, wherein the dispenser is configured to open when mechanically pressed and otherwise to stay closed.

24. The dispensing mechanism of claim 20,
wherein the volume modifier comprises a piston configured to define, together with a housing in which the dispensing mechanism is arranged, a moving chamber, and
wherein the expandable buffering reservoir is coupled to one side of the piston and arranged in the moving chamber such that moving the piston longitudinally relative to the housing causes modifying the volume of the expandable buffering reservoir.

25. The dispensing mechanism of claim 24, wherein the dispensing reservoir is coupled to another side of the piston opposite to the moving chamber, causing the dispensing reservoir to be moved together with the piston longitudinally in the housing.

26. The dispensing mechanism of claim 20, wherein the dispensing reservoir comprises a deformable opening configured as a flexible baby bottle teat valve or as a flexible duckbill valve.

27. The dispensing mechanism of claim 20, wherein the dispensing reservoir is configured to be deformable.

28. The dispensing mechanism of claim 20, wherein the dispensing reservoir is made from silicon.

29. The dispensing mechanism of claim 20, wherein the dispensing reservoir has a volume of 0.4 microliters to 0.5 microliters.

30. The dispensing mechanism of claim 24, wherein the piston comprises the outlet valve connecting the outlet of the expandable buffering reservoir to the inlet of the dispensing reservoir.

31. The dispensing mechanism of claim 22, wherein the wherein the volume modifier comprises a piston, and wherein the biasing member is a compression spring attached to the piston.

32. The dispensing mechanism of claim 20, wherein the expandable buffering reservoir is accordion-shaped, and wherein modifying a volume of the expandable buffering reservoir comprises adjusting a length of the expandable buffering reservoir.

33. The dispensing mechanism of claim 20, further comprising a chained arrangement of one or more expandable drawing reservoirs, wherein the one or more expandable drawing reservoirs are arranged between the expandable buffering reservoir and the dispensing reservoir.

34. The dispensing mechanism of claim 33, wherein each of the one or more expandable drawing reservoirs is connected to a neighboring reservoir by a one-way valve in such a way that the liquid only flows from the expandable buffering reservoir to the dispensing reservoir.

35. An aerosol-generating article, comprising:
a liquid storage portion configured to store pressurized liquid;
a vaporizer configured to vaporize liquid;
a dispensing mechanism connecting the liquid storage portion to the vaporizer, the dispensing mechanism comprising:
an expandable buffering reservoir with an inlet and an outlet;
a dispensing reservoir of constant volume with an inlet and an outlet;
an inlet valve connected to the inlet of the expandable buffering reservoir and being configured to be connected to the liquid storage portion filled with pressurized liquid such that liquid can only flow from the liquid storage portion to the expandable buffering reservoir;
an outlet valve connecting the outlet of the expandable buffering reservoir to the inlet of the dispensing reservoir such that the liquid can only flow from the expandable buffering reservoir to the dispensing reservoir; and
a volume modifier configured to modify a volume of the expandable buffering reservoir,
wherein expanding a current volume of the expandable buffering reservoir to an expanded volume causes a drawing of liquid from the liquid storage portion to the expandable buffering reservoir until the expanded volume of the expandable buffering reservoir is filled up with the liquid,
wherein compressing the expanded volume of the expandable buffering reservoir causes an expelling of the liquid from the expandable buffering reservoir to the dispensing reservoir until the dispensing reservoir is filled up with the liquid, and
wherein the dispensing reservoir is configured to release a content of the liquid during one puff of a user; and
a housing having at least one air inlet and at least one air outlet,
wherein the vaporizer is further configured to receive the liquid from the outlet of the dispensing reservoir.

36. The aerosol-generating article of claim 35, wherein the volume modifier is further configured to expand the current volume of the expandable buffering reservoir to the expanded volume upon the user drawing air into the housing.

37. The aerosol-generating article of claim 36, wherein the housing comprises at least one housing stop configured to press or squeeze a dispenser connected to the outlet of the dispensing reservoir when the dispensing reservoir moves longitudinally relative to the housing towards the at least one housing stop upon the volume modifier expanding the volume of the expandable buffering reservoir.

38. An aerosol-generating system, comprising:
an aerosol-generating article, comprising:
a liquid storage portion configured to store pressurized liquid;
a vaporizer configured to vaporize liquid;
a dispensing mechanism connecting the liquid storage portion to the vaporizer, the dispensing mechanism comprising:
an expandable buffering reservoir with an inlet and an outlet;
a dispensing reservoir of constant volume with an inlet and an outlet;
an inlet valve connected to the inlet of the expandable buffering reservoir and being configured to be connected to the liquid storage portion filled with pressurized liquid such that liquid can only flow from the liquid storage portion to the expandable buffering reservoir;
an outlet valve connecting the outlet of the expandable buffering reservoir to the inlet of the dispensing reservoir such that the liquid can only flow from the expandable buffering reservoir to the dispensing reservoir; and
a volume modifier configured to modify a volume of the expandable buffering reservoir,
wherein expanding a current volume of the expandable buffering reservoir to an expanded volume causes a drawing of liquid from the liquid storage portion to the expandable buffering reservoir until the expanded volume of the expandable buffering reservoir is filled up with the liquid,
wherein compressing the expanded volume of the expandable buffering reservoir causes an expelling of the liquid from the expandable buffering reservoir to the dispensing reservoir until the dispensing reservoir is filled up with the liquid, and
wherein the dispensing reservoir is configured to release a content of the liquid during one puff of a user; and
a housing having at least one air inlet and at least one air outlet,
wherein the vaporizer is further configured to receive the liquid from the outlet of the dispensing reservoir; and
a mouthpiece configured to deliver vaporized liquid from the vaporizer to the user that draws into the mouthpiece.
